(19) United States
(12) Patent Application Publication
(10) Pub. No.: US 2026/0364536 A1
(43) Pub. Date: Nov. 19, 2026

(54) METHODS, MATERIALS, AND SYSTEMS FOR CONCENTRATING BIOMOLECULES

(71) Applicants: ARIZONA BOARD OF REGENTS ON BEHALF OF ARIZONA STATE UNIVERSITY, Scottsdale, AZ (US); THE PENN STATE RESEARCH FOUNDATION, University Park, PA (US)

(72) Inventors: Marylaura Lind Thomas, Scottsdale, AZ (US); Andrew Zydney, University Park, PA (US)

(21) Appl. No.: 19/673,447

(22) Filed: May 11, 2026

Related U.S. Application Data
(60) Provisional application No. 63/805,441, filed on May 14, 2025.

(51) Int. Cl. B01D 61/36; B01D 69/02; B01D 69/12; B01D 71/28; B01D 71/80; C07K 1/34; C07K 16/00

(52) U.S. Cl. CPC B01D 61/362; B01D 69/02; B01D 69/12; B01D 71/281; B01D 71/80; C07K 1/34; C07K 16/00; B01D 2315/12; B01D 2325/04

(57) ABSTRACT

Provided herein are methods for concentrating a biopharmaceutical feed stream comprising a biomolecule. The methods comprise contacting a first side of a dense, non-porous membrane with the biopharmaceutical feed stream, wherein the feed stream comprises at least one biomolecule and a solvent, maintaining a chemical potential gradient across the membrane such that at least a portion of the solvent is transported through the membrane via a solution-diffusion mechanism and exits a second side of the membrane as a vapor permeate, and recovering a retentate stream from the first side of the membrane, wherein the retentate stream comprises the biomolecule at a concentration higher than that of the feed stream. Related methods, systems, thin-film composite pervaporation membranes materials, and concentrated biopharmaceutical formulations are also provided.

### CROSS-REFERENCE TO RELATED APPLICATIONS

This application claims priority to U.S. Provisional Patent Application Ser. No. 63/805,441, filed May 14, 2025, the disclosure of which is incorporated herein by reference.

### BACKGROUND

Biopharmaceutical products are molecules produced by living organisms and used as medicines. Some examples of biopharmaceutical products are highly active hormones (e.g., insulin), thrombolytic agents (e.g., tissue-type plasminogen activator), clotting factors (e.g., Factor VIII), and monoclonal antibodies (mAbs) used for the treatment of various diseases (e.g., Herceptin® for the treatment of breast cancer and Rituxan® for the treatment of B-cell lymphoma). Molecules like mAbs act stoichiometrically, binding to a particular receptor or cell type, requiring much higher dosing levels and batch sizes. It is essential to note that biopharmaceuticals, for most applications, are typically not one-off "cures" but rather maintenance therapies for the management of chronic and terminal conditions. The demand for biologically derived products, including both recombinant proteins and nucleic acid therapeutics, is steadily increasing; BioSpace estimates that the overall biopharmaceutical market size is $0.5 trillion in 2025 and projected to reach $1.7 trillion by 2034.

To further take mAbs as an exemplary illustration, according to a 2023 report, the global market size of mAbs was ~$215B (~50% of which is spent in North America). The largest mAb by market size is Merck's Keytruda, used for the treatment of metastatic melanoma. Merck reported sales of ~$29.5B in 2024. It is delivered as an intravenous infusion at a concentration of 25 mg/mL to achieve the desired therapeutic response and was recently (Oct 2025) approved by the U.S. Food and Drug Administration (FDA) for subcutaneous injection. The potential future size of the mAb market is much larger than the current size. Many mAb-based therapeutics are being studied for applications in Alzheimer's, hypercholesterolemia (with millions of potential patients), and the treatment of infectious diseases (e.g., Covid-19 and its variants, or influenza).

The quantity of mAb required to achieve a therapeutic effect in a patient is disorder-dependent. The following are typical therapeutic dose ranges: (1) the low end: where 20 mg of ixekizumab (Taltz®) is given via a 0.25 mL subcutaneous injection every 2-4 weeks to treat moderate-to-severe plaque psoriasis (market size $3.3B), and (2) the high end: where 1200 mg of atezolizumab (Tecentriq®) is given via a 20 mL intravenous infusion every 2-4 weeks to treat Non-Small Cell Lung Cancer (market size $4.5B). Subcutaneous volumes are limited to <1.5 mL per injection. Therefore, many mAb therapeutics, which need to be delivered at dosages > 190 mg, are currently delivered through intravenous infusions in the clinic, in large part because of the difficulty in achieving the highly concentrated formulations that would be required for subcutaneous injection.

Recent clinical trials of several mAbs for treating influenza infections used an average dose of 3400 mg, delivered by intravenous infusion, because the quantity of therapeutic material is too large for subcutaneous injection with current manufacturing methods. The successful large-scale commercialization of mAbs for viral infection treatment is unlikely with intravenous infusion, as this can only be done in hospitals or outpatient clinics.

If the mAb therapeutic can be delivered via subcutaneous injection, after the initial intravenous induction dose is administered in a clinical setting to monitor for adverse reactions, subsequent subcutaneous injection maintenance doses can usually be administered by the patient themselves (or by a caregiver to the patient) at home. However, if the mAb therapeutic can only be delivered by intravenous infusion, the patient must repeatedly return to a clinical infusion center to receive subsequent maintenance doses, which is burdensome and expensive.

FIG. 1 provides a high-level overview of the sequential steps involved in mAb production. Stage 1 involves the synthesis of the product by genetically engineered cells, most commonly Chinese hamster ovary (CHO) cells in the case of mAbs. Stage 2 is the harvest stage, also referred to as recovery; in this stage, the desired product (mAb) is separated from the bulk cells that produced it into a clarified aqueous solution, often through centrifugation, membrane microfiltration, or depth filtration. In Stage 3, the purification stage, the mAb product is purified from other soluble impurities present in the aqueous solution. Typically, the unit operations used for purification in Stage 3 are column chromatography, with Protein A affinity chromatography the method of choice for most mAbs, commonly followed by ion exchange and mixed-mode resins. Stage 4 is the final stage of formulation and filling. The objective of Stage 4 is to produce the final dosage and to ensure acceptable quality attributes related to appearance, quantity, potency, and safety. Stage 4 includes ensuring the purified mAb product in liquid solution from Stage 3 is at the correct concentration, is in the correct buffer system, and includes the proper excipients (in addition to the buffering agents). Formulation is necessary because it is likely that the product concentration and buffer in the effluent from Stage 3 are not what is required for the final drug product, but rather, are dictated by the final chromatography step in the process.

Ultrafiltration has been the method of choice for concentrating recombinant proteins and mAbs in the biopharmaceutical industry. Ultrafiltration uses applied hydraulic pressure to drive water/buffer through a semipermeable membrane while retaining the product of interest. Primarily, this is achieved through batch ultrafiltration, in which the mAb solution is recirculated through the membrane module. At the same time, water and buffer components are driven through the membrane under applied hydraulic pressure, thereby concentrating the mAb solution.

The maximum achievable concentration of mAb materials obtained by ultrafiltration is generally less than 190 mg/mL, which is often lower than the desired target concentration for formulation and filling; this is particularly true when considering the need to over-concentrate solutions to accommodate post-ultrafiltration dilution during product recovery and/or the addition of excipients. This maximum achievable concentration of solutions is a fundamental limitation of the ultrafiltration process - it cannot achieve very high product concentrations because of 1) mechanical limitations on the maximum tolerable trans-membrane pressure associated with the membrane/module, 2) high viscosity of the concentrated mAb solution (which also contributes to high pressures), and 3) membrane fouling. An additional limitation of current processes is that many ultrafiltration systems are single-use; the membranes are difficult to clean, and it is rare for companies to reuse a bioprocessing membrane for more than 10-20 production cycles.

One of the challenges in producing these biotherapeutics is the high concentrations often required to deliver an effective dose of the drug in a single subcutaneous injection. Many antibodies are currently formulated at concentrations exceeding 200 mg/mL. These higher concentrations are often achieved using time-consuming and expensive thermal drying processes such as lyophilization, in which case it is then necessary to resolubilize the mAb prior to administration, which can create additional challenges for self-administration by subcutaneous injection.

Accordingly, there is a need for additional methods, and related aspects, of concentrating biomolecules, including monoclonal antibodies.

### SUMMARY

The present disclosure provides a pervaporation system and related aspects for concentrating various types of biomolecules, including biopharmaceutical mAbs. In many implementations, for example, the pervaporation technology disclosed herein can be directly incorporated into Stage 4 biomolecule production. The disclosed technology surpasses the current solution concentration limitations of other processes, such as ultrafiltration. These and other attributes of the present disclosure will be apparent to those of ordinary skill in the art upon a complete review of this specification, including the accompanying figures.

In one aspect, the present disclosure provides a method for concentrating a biopharmaceutical feed stream comprising a biomolecule. The method includes (a) contacting a first side of a dense, non-porous membrane with the biopharmaceutical feed stream, wherein the feed stream comprises at least one biomolecule and a solvent; (b) maintaining a chemical potential gradient across the membrane such that at least a portion of the solvent is transported through the membrane via a solution-diffusion mechanism and exits a second side of the membrane as a vapor permeate; and (c) recovering a retentate stream from the first side of the membrane, wherein the retentate stream comprises the biomolecule at a concentration higher than that of the feed stream.

In another aspect, the present disclosure provides a system for the concentration of biopharmaceutical products. The system comprises a feed reservoir containing a feed stream comprising a biomolecule at an initial concentration; a pervaporation module comprising a dense, non-porous membrane separating a feed side from a permeate side; a vacuum source in fluid communication with the permeate side of the membrane configured to maintain a vapor pressure differential across the membrane; and a condenser configured to capture solvent vapor exiting the permeate side; wherein the system is configured to concentrate the biomolecule to a final concentration of at least 200 mg/mL at a volumetric flux of at least 0.1 L m⁻² h⁻¹.

In another aspect, the present disclosure provides a thin-film composite pervaporation membrane specifically configured for biopharmaceutical concentration, comprising: (a) a porous support layer comprising polysulfone or polyethersulfone; and (b) a dense, non-porous active layer disposed on the support layer, the active layer comprising a sulfonated pentablock copolymer; wherein the membrane is characterized by the ability to retain >99% of a monoclonal antibody in a feed stream while maintaining a solvent flux of at least 1.0 L m⁻² h⁻¹ under a vacuum of -0.9 bar.

### Definitions

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, reaction mixtures, compositions, materials, devices, kits, and systems, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer : As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody : As used herein, the term “antibody” refers to an immunoglobulin or an antigen-binding domain thereof. The term includes but is not limited to polyclonal, monoclonal, monospecific, polyspecific, non-specific, bispecific, humanized, human, caninized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a target analyte, e.g., a molecule in which one or more immunoglobulin chains is not full length, but which specifically binds to a target analyte. Examples of binding portions encompassed within the term “antigen-binding portion of an antibody include (i) a Fab fragment, a monovalent fragment consisting of the VLC, VHC, CL and CH1 domains: (ii) a F(ab')2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV)). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Biomolecule: As used herein, the term "biomolecule" refers to any molecule, or a functional fragment or derivative thereof, that is produced by, found in, or otherwise associated with a living organism. For the purposes of this disclosure, "biomolecule" expressly includes: (1) molecules produced in vivo by a biological system (e.g., a cell, tissue, or multicellular organism); (2) molecules produced in vitro or in a cell-free environment (e.g., through enzymatic synthesis or biochemical reactions outside of a living cell); and (3) molecules produced synthetically or semi-synthetically (e.g., via chemical synthesis), provided the molecule possesses a structure or sequence substantially similar to a molecule found in nature. The term "biomolecule" encompasses, but is not limited to, the following classes of compounds: polypeptides and proteins (e.g., enzymes, antibodies, receptors, and signaling peptides); nucleic acids (e.g., DNA, RNA, oligonucleotides, and aptamers, whether single-stranded or double-stranded); carbohydrates and polysaccharides (e.g., monosaccharides, oligosaccharides (such as human milk oligosaccharides or HMOs), and complex glycans); lipids (e.g., phospholipids, glycolipids, steroids, and fatty acids); and metabolites (e.g., small molecule organic compounds produced during cellular metabolism). Any reference to a specific biomolecule herein shall be understood to encompass variants, analogs, and modified versions of that biomolecule (e.g., pegylated, glycosylated, or fluorophore-conjugated forms) unless otherwise specified.

Biopharmaceutical: As used herein, the term “biopharmaceutical” refers to a biomolecule that is used as a medicine.

Biopharmaceutical Feed Stream: As used herein, the term “biopharmaceutical feed stream” refers to a solution containing molecules produced by living organisms for medicinal use. This includes, but is not limited to, recombinant proteins and monoclonal antibodies (mAbs).

Continuous Flow-Through / Feed-and-Bleed: As used herein, the terms “continuous flow-through“ and “feed-and-bleed” refer to industrial membrane system configurations. A "continuous" process involves a steady flow of feed and removal of product, while a "feed-and-bleed" configuration involves recirculating a portion of the retentate to the feed side to achieve high concentration factors.

Membrane : As used herein, the term “membrane” refers to a permselective barrier or interface between two phases; phase I is usually considered the feed or upstream side phase while phase 2 is considered the permeate or downstream side. Separation is achieved because the membrane has the ability to transport one component from the feed mixture more readily than any other component or components. The driving force for transport across a membrane may be a chemical potential difference, a pressure difference, a temperature difference, a concentration difference, a heat difference, a volume difference, a momentum difference, an electrical potential difference, or an electrochemical difference.

Non-Volatile Excipient or Buffer: As used herein, the term “non-volatile excipient or buffer” refers to formulation components (e.g., histidine, citrate, sodium chloride, or polysorbate 80) that have essentially no vapor pressure at the operating temperatures (e.g., 20-24 ºC) and pressures of the process and are therefore retained in the retentate along with the biomolecules.

Retentate Stream: As used herein, the term “retentate stream” refers to the portion of the feed stream that does not pass through the membrane and is recovered from the feed side of the pervaporation module. In the context of this disclosure, the retentate stream contains the concentrated biomolecules.

Sample: As used herein, the term “sample” means anything capable of being analyzed by the methods, devices, and/or systems disclosed herein.

Subcutaneous Injection: As used herein, the term “subcutaneous injection” refers to the administration of a drug into the layer of fat between the skin and the muscle, characterized by a limited delivery volume, typically less than 1.5 mL per injection.

Subject: As used herein, the term “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, the term "system" in the context of analytical instrumentation refers a group of objects and/or devices that form a network for performing a desired objective.

Treat: As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Vapor Permeate: As used herein, the term “vapor permeate” refers to the solvent (e.g., water) that has been transported across the membrane and exists in a gaseous state on the permeate side before being captured, for example, by a condenser.

Volumetric Flux: As used herein, the term “volumetric flux” refers to a metric representing the volume of permeate passing through a unit area of membrane per unit of time (e.g., L m⁻² h⁻¹ or kg m⁻² h⁻¹).

Water Permeance: As used herein, the term “water permeance” refers to the volumetric flux normalized by the driving force (the vapor pressure differential across the membrane). This metric (expressed in L m⁻² h⁻¹ bar⁻¹) allows for the comparison of membrane performance across different operating conditions.

### BRIEF DESCRIPTION OF THE DRAWINGS

The accompanying drawings, which are incorporated in and constitute a part of this specification, illustrate certain embodiments, and together with the written description, serve to explain certain principles of the methods, uses, kits, devices, systems, and related compositions disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a schematic that provides a high-level overview of the sequential steps involved in mAb production. As shown, Stage 1 is the product synthesis from cellular material. Stage 2 is the harvest stage. Stage 3 is the purification stage. Stage 4 is the final formulation/filling stage.

FIG. 2 is a flowchart that shows an exemplary method for concentrating a biopharmaceutical feed stream comprising a biomolecule in accordance with an embodiment.

FIG. 3 is a schematic diagram of an exemplary system suitable for use with certain aspects disclosed herein.

FIGS. 4A-4F are data plots showing a performance comparison of ultrafiltration, forward osmosis, and pervaporation. FIGS. 4A and 4D are plots of ultrafiltration data using immunoglobulin G showing that ~150 mg/mL ultrafiltration fails through limitations of fouling, viscosity, and pressure; membrane permeance with driving force of ~2.25 bar applied hydraulic pressure. FIGS. 4B and 4E are plots of forward osmosis data with immunoglobulin G and polyethylene glycol; forward osmosis concentrates solutions past ultrafiltration; membrane permeance with ~49 bar of osmotic pressure. Polyethylene glycol is less viscous than immunoglobulin G which accounts for faster flux decline with immunoglobulin G feeds. FIGS. 4C and 4F are plots of data using commercially available pervaporation membranes with polyethylene glycol and IgG feeds.

FIG. 5 is a table showing membrane key performance equations.

FIG. 6A is a photo of a lab-scale, cross-flow pervaporation module with a membrane area of 15.63 cm².

FIG. 6B is a schematic of an exemplary cross-flow pervaporation system showing peristaltic feed pump, the membrane module, the condenser to capture permeate, and adjustable vacuum level to create the vapor pressure differential across the dense pervaporation membrane.

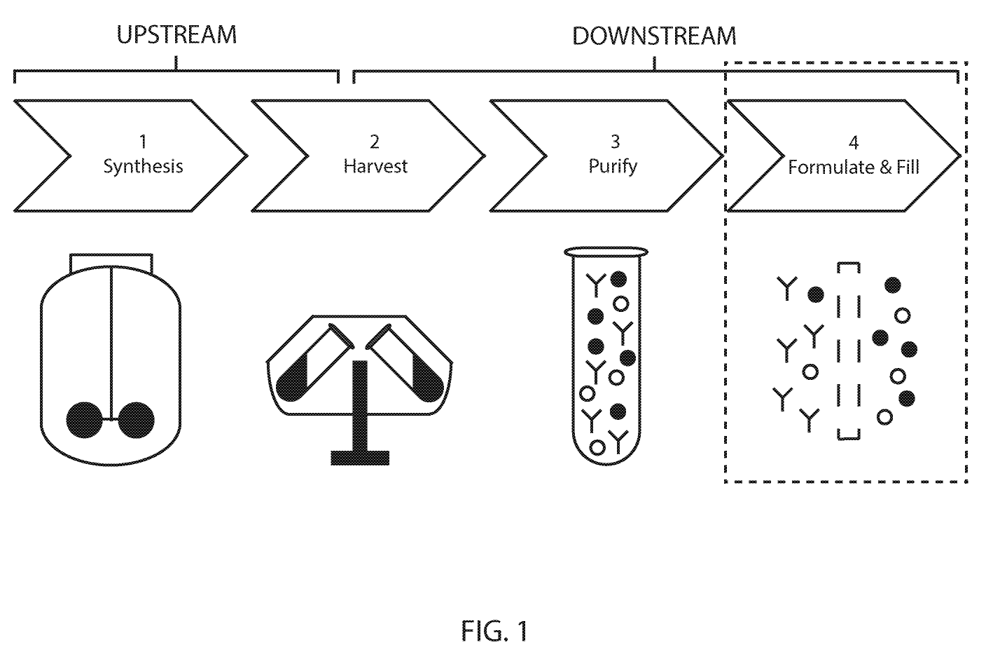

### DETAILED DESCRIPTION

### I. Introduction

The following detailed description is provided to enable a person of ordinary skill in the art to make and use the subject matter described herein and is not intended to limit the scope of the claimed subject matter in any manner. It should be understood that the embodiments and examples described below are illustrative and that variations and modifications may be made without departing from the spirit and scope of the present disclosure as defined by the appended claims.

In various embodiments, the present disclosure provides a pervaporation unit operation that can be directly incorporated into Stage 4 biomolecule production (e.g., monoclonal antibodies (mAbs), recombinant proteins, nucleic acids, peptide nucleic acids, and the like) providing a platform technology that surpasses the current solution concentration limitations of, for example, gold standard ultrafiltration processes. In some embodiments, pervaporation is used in Stage 4 biopharmaceutical production to increase the concentration of purified biomolecules within formulation solutions. Although much of the present disclosure focuses on pervaporation in the context of mAb production for purpose of clarity of illustration, it will be appreciated that the technology disclosed herein can be applied to the production of essentially any biomolecule.

By way of further background, pervaporation (pervap or PV) is a membrane process that uses dense polymer membranes (or molecular sieving membranes) with transport driven by the difference in chemical potential. This driving force is created by applying one or both of: a pressure differential (keeping feed at atmospheric pressure and permeate at vacuum) or a temperature differential (heating feed to no more than the boiling point). The resulting pressure or temperature differentials across the membrane create a vapor pressure difference, which is the driving force that causes transport from the feed to the permeate across the membrane. A key benefit of pervaporation is that its driving force, the cross-membrane water vapor differential, does not suffer from concentration polarization (e.g., decline as the concentration of solute increases in the feed), unlike pressure-driven membrane processes.

To further illustrate, lyophilization (e.g., freeze-drying) and forward osmosis can provide high-concentration formulations; however, they also have drawbacks such as increasing the difficulty of resolubilization or requiring complicated operating procedures.

Because biopharmaceuticals, such as mAbs, have essentially no vapor pressure (e.g., similar to sodium chloride) at the room temperatures and pressures at which the pervaporation process disclosed herein operates, there is essentially no mAb transport across the membrane out of the retentate (the concentrated feed during the process). It is important to note that, in pervaporation, other components in the feed that do not have a high vapor pressure at the operating conditions are also retained in the concentrated feed (e.g., buffers such as histidine and non-volatile excipients like polysorbate 80). Pervaporation has two key differentiators compared to state-of-the-art ultrafiltration: 1) pervaporation uses a dense, nonporous membrane and operates on a solution-diffusion-based separation mechanism, and 2) it can achieve very high concentration factors in a single-step process. These principles allow the system to operate gently, with minimal stress on biomolecules, while efficiently handling high-concentration feeds without the fouling issues associated with porous ultrafiltration membranes. Pervaporation also avoids the complex draw solution and draw recovery process required by forward osmosis.

Producing mAb and other biopharmaceutical drugs at concentrations of >200 mg/mL is limited by current biopharmaceutical ultrafiltration-based manufacturing processes. In some embodiments, the technology disclosed herein provides readily integrated processes to: 1) reduce biopharmaceutical manufacturing costs (and subsequent consumer costs), and 2) enable simple manufacturing of biopharmaceuticals, such as mAbs at concentrations of >200 mg/mL to enable delivery by at-home subcutaneous injection. These and other attributes will be apparent upon a complete review of the present disclosure, including the accompanying figures.

### II. Exemplary Embodiments

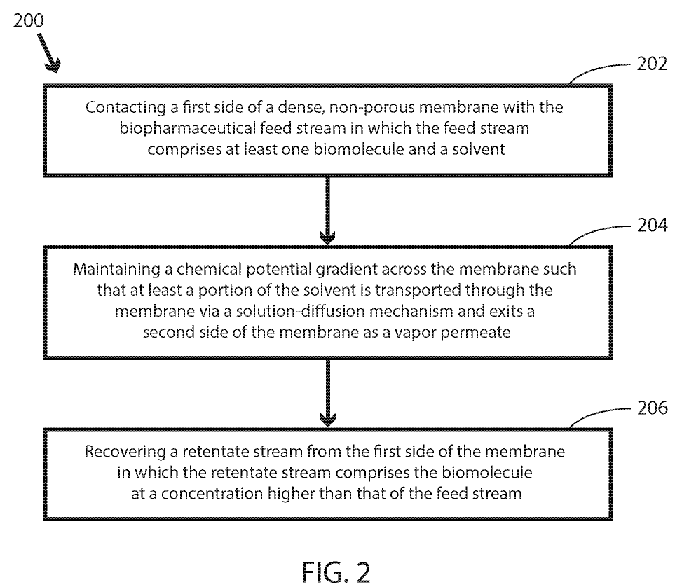

To illustrate, FIG. 2 is a flowchart that shows an exemplary method for concentrating a biopharmaceutical feed stream comprising a biomolecule. As shown, method 200 includes contacting a first side of a dense, non-porous membrane with the biopharmaceutical feed stream, wherein the feed stream comprises at least one biomolecule and a solvent (step 202). In some embodiments, the biopharmaceutical feed stream further comprises at least one non-volatile excipient or buffer, and method 200 further comprises retaining the non-volatile excipient or buffer in the retentate stream alongside the concentrated biomolecule. In some embodiments, the biomolecule comprises a monoclonal antibody (mAb), a recombinant protein, or a nucleic acid. Method 200 also includes maintaining a chemical potential gradient across the membrane such that at least a portion of the solvent is transported through the membrane via a solution-diffusion mechanism and exits a second side of the membrane as a vapor permeate (step 204). In some embodiments, the chemical potential gradient is maintained by applying a vacuum to the second side of the membrane to create a vapor pressure differential.

In addition, method 200 also includes recovering a retentate stream from the first side of the membrane, wherein the retentate stream comprises the biomolecule at a concentration higher than that of the feed stream (step 206). In some embodiments, the concentration of the biomolecule in the retentate stream is greater than 200 mg/mL, preferably greater than 300 mg/mL, and more preferably up to 600 mg/mL. In some embodiments, method 200 further comprises a pre-concentration step using ultrafiltration or diafiltration prior to the pervaporation contacting step. In some embodiments, method 200 is performed as a continuous flow-through process or a feed-and-bleed process.

Various membrane materials are optionally used in performing method 200. In some embodiments, for example, the dense, non-porous membrane comprises a sulfonated pentablock copolymer. In some embodiments, the sulfonated pentablock copolymer comprises polyisoprene, poly(t-butyl styrene), polystyrene, and poly(sulfonated styrene). In some embodiments, the membrane is a thin-film composite (TFC) membrane comprising an active selective layer having a thickness between 100 nm and 20 µm disposed on a porous support material. In some embodiments, the dense, non-porous membrane exhibits a water permeance of at least 300 L m⁻² h⁻¹ bar⁻¹.

To further illustrate, in some embodiments, the present disclosure provides a thin-film composite pervaporation membrane specifically configured for biopharmaceutical concentration. The membrane comprises a porous support layer comprising polysulfone or polyethersulfone and a dense, non-porous active layer disposed on the support layer, the active layer comprising a sulfonated pentablock copolymer. The membrane is characterized by the ability to retain >99% of a monoclonal antibody in a feed stream while maintaining a solvent flux of at least 1.0 L m⁻² h⁻¹ under a vacuum of -0.9 bar.

The present disclosure also provides various concentrated biopharmaceutical formulations produced by method 200. In some embodiments, such a concentrated biopharmaceutical formulation comprises a monoclonal antibody and/or other biomolecule at a concentration of 200 mg/mL to 600 mg/mL and at least one concentrated excipient, wherein the formulation is suitable for subcutaneous injection.

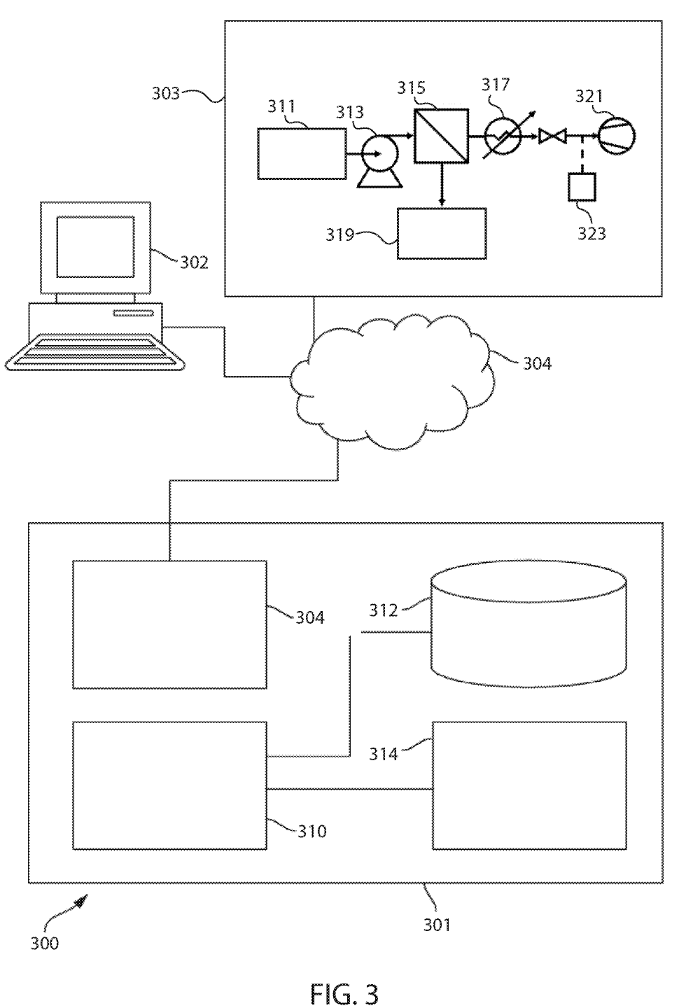

In some embodiments, the present disclosure also provides various systems and related computer readable media of use in implementing the methods disclosed herein. To illustrate, FIG. 3 is a schematic diagram of a hardware computer system 300 suitable for implementing various embodiments. For example, FIG. 3 illustrates various hardware, software, and other resources that can be used in implementations of any of the methods disclosed herein, including method 200. System 300 includes controller terminal 302 and computer 301. Terminal 302 and computer 301 may be communicatively coupled by way of one or more networks 304, e.g., the internet. In some embodiments, controller terminal 302 and computer 301 together comprise processing and computation modules of system 300.

Computer 301 may be implemented as any of a desktop computer, a laptop computer, can be incorporated in one or more servers, clusters, or other computers or hardware resources, or can be implemented using cloud-based resources. Computer 301 includes volatile memory 314 and persistent memory 312, the latter of which can store computer-readable instructions, that, when executed by electronic processor 310, configure computer 301 to perform any of the methods disclosed herein, including method 200. Computer 301 further includes network interface 308, which communicatively couples computer 301 to terminal 302 and to sub-system 303 (described further herein) via network 304. Other configurations of system 300, associated network connections, and other hardware, software, and service resources are possible. In some embodiments, sub-system 303 is implemented as a stand-alone system for the concentration of biopharmaceutical products using the methods disclosed herein.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

As shown, sub-system 303 is configured for the concentration of biopharmaceutical products. As shown, sub-system 303 includes feed reservoir 311 containing a feed stream comprising a biomolecule at an initial concentration and pervaporation module 315 comprising a dense, non-porous membrane separating a feed side from a permeate side. In some embodiments, the dense, non-porous membrane exhibits a water permeance of at least 300 L m⁻² h⁻¹ bar⁻¹. Pump 313 conveys the feed stream from feed reservoir 311 to pervaporation module 315 via an operably connected fluid conduit. Sub-system 303 also includes vacuum source 321 in fluid communication with the permeate side of the membrane configured to maintain a vapor pressure differential across the membrane. The vapor pressure differential is measured by pressure gauge 323. In addition, sub-system 303 also includes condenser 317 configured to capture solvent vapor exiting the permeate side. The retentate is collected in retentate reservoir 319. Sub-system 303 is configured to concentrate the biomolecule to a final concentration of at least 200 mg/mL at a volumetric flux of at least 0.1 L m⁻² h⁻¹. In some embodiments, sub-system 303 further comprises an ultrafiltration unit positioned upstream of and in fluid communication with the pervaporation module.

### III. EXAMPLES

### Example 1: Concentration of PEG and IgG Solutions

PEG and IgG solutions were used to evaluate the basic performance of the pervaporation system. The commercially available membrane to be used for conducting mAb concentration tests was tested (commercial name of the polymer: NEXAR^{TM}).

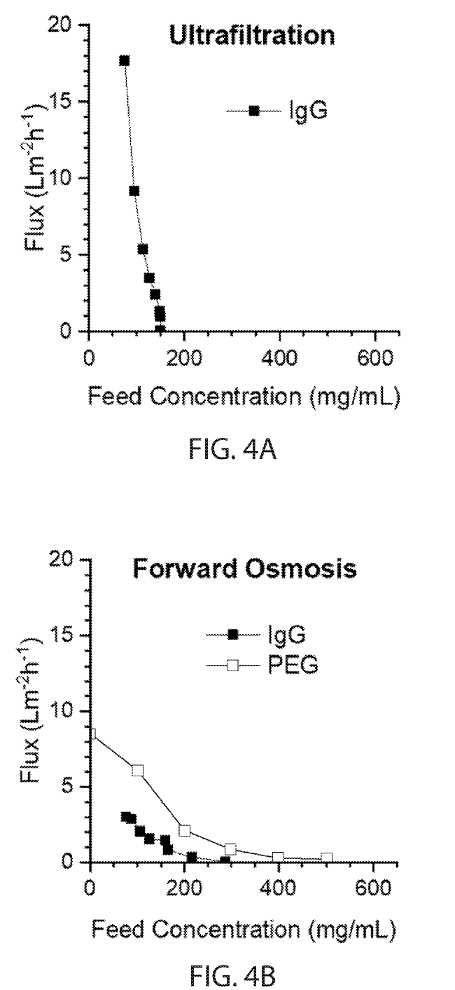

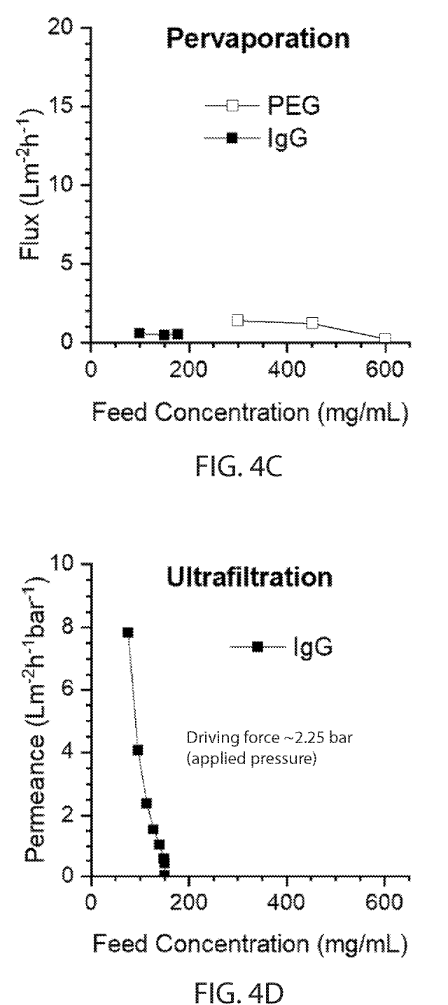

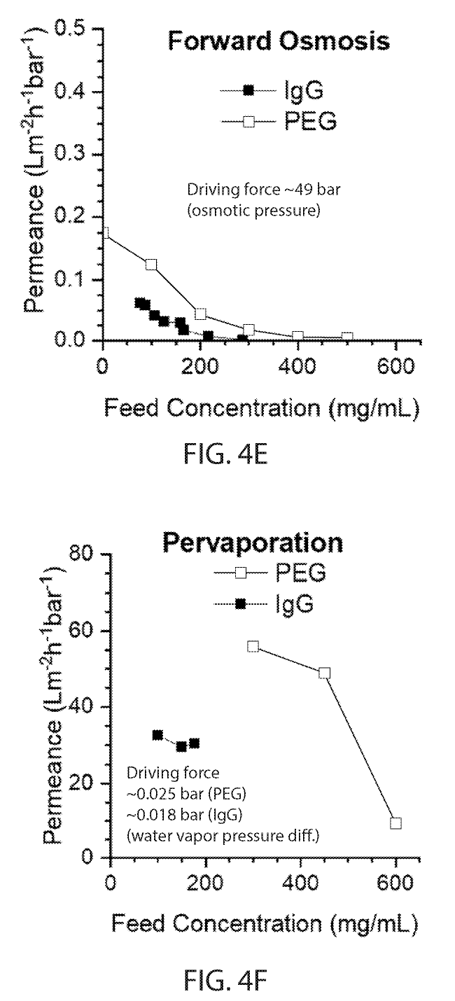

FIGS. 4A and 4B are data plots showing that forward osmosis can reach higher concentrations of polyethylene glycol and immunoglobulin G than ultrafiltration. FIG. 4C displays data indicating that pervaporation outperforms both ultrafiltration and forward osmosis in concentrating polyethylene glycol solutions. As seen in FIGS. 4A and 4B, the flux in ultrafiltration of immunoglobulin G drops precipitously to 0 at feed concentrations > 150 mg/mL due to transmembrane pressure limitations. It has been observed that forward osmosis maintains fluxes of ~ 1.7 L m⁻² h⁻¹ for feed concentrations of 150 mg/mL and maintains fluxes of ~ 0.08 L m⁻² h⁻¹ for feed concentrations up to 290 mg/mL. Also, data show fluxes of up to ~ 1.4 L m⁻² h⁻¹ for polyethylene glycol feeds of 300 mg/mL (recirculating, vacuum operation) and fluxes of up to ~ 0.5 L m⁻² h⁻¹ for IgG feeds of 177 mg/mL (no recirculation, sweeping gas operation).

### Example 2: Membrane Synthesis

The hydrophilic commercial polymer NEXAR^{TM} is used for the membrane selective layer. NEXAR^{TM} has a low water contact angle, ranging from 50° to 70°. NEXAR^{TM} is relatively inexpensive, at approximately $2/kg (for comparison, a material like Nafion costs $6000/kg). Furthermore, NEXAR^{TM} is a green alternative to fluorinated and perfluorinated polymers, which are frequently used in membrane technology. NEXAR^{TM} is a sulfonated pentablock copolymer of polyisoprene, poly(t-butyl styrene), polystyrene, and poly(sulfonated styrene); the U.S. Food and Drug Administration (US FDA) has approved each of these homopolymers for use in food, pharmaceuticals, or devices.

NEXAR^{TM} has excellent performance for pervaporation desalination (transporting water and retaining sodium chloride), with membranes achieving water permeances of 120 L m⁻² h⁻¹ bar⁻¹. Membrane synthesis can be further optimized to achieve membranes with permeances over 200 L m⁻² h⁻¹ bar⁻¹, which enable the pervaporation membrane and unit operation to have similar (or better) performance and capital footprint compared to existing membrane mAb production processes, while allowing much higher final mAb formulation concentrations due to the inherent advantages of pervaporation technology.

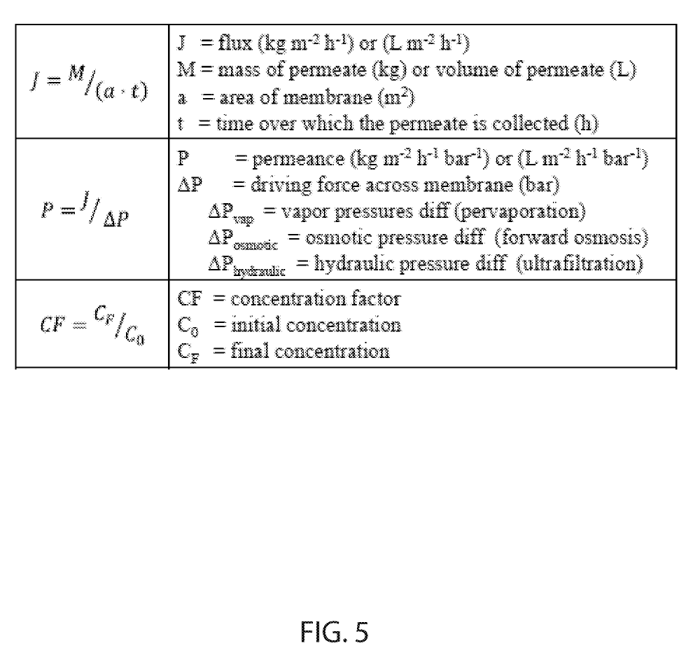

A methodology is developed for casting NEXAR^{TM} onto commercially available porous polysulfone support membranes, similar to standard methodologies in membrane science for creating thin film composite membranes (e.g., from Sterlitech). Thin film composite membranes consist of a thin active layer (ranging from ~ 100 nm to 20 µm in thickness) deposited onto a porous support material (e.g., polysulfone); the active layer is responsible for the separation properties of the membrane, while the support material provides mechanical support to the system enabling scaleable roll-to-roll production and packaging into larger modules (e.g., spiral wound modules or large flat sheet modules). Since flux (as defined in FIG. 5) is roughly inversely proportional to membrane thickness, the membrane is made with a NEXAR^{TM} layer ~10-30 µm thick to ensure a fast dehydration process.

### Example 3: Lab-Scale Testing

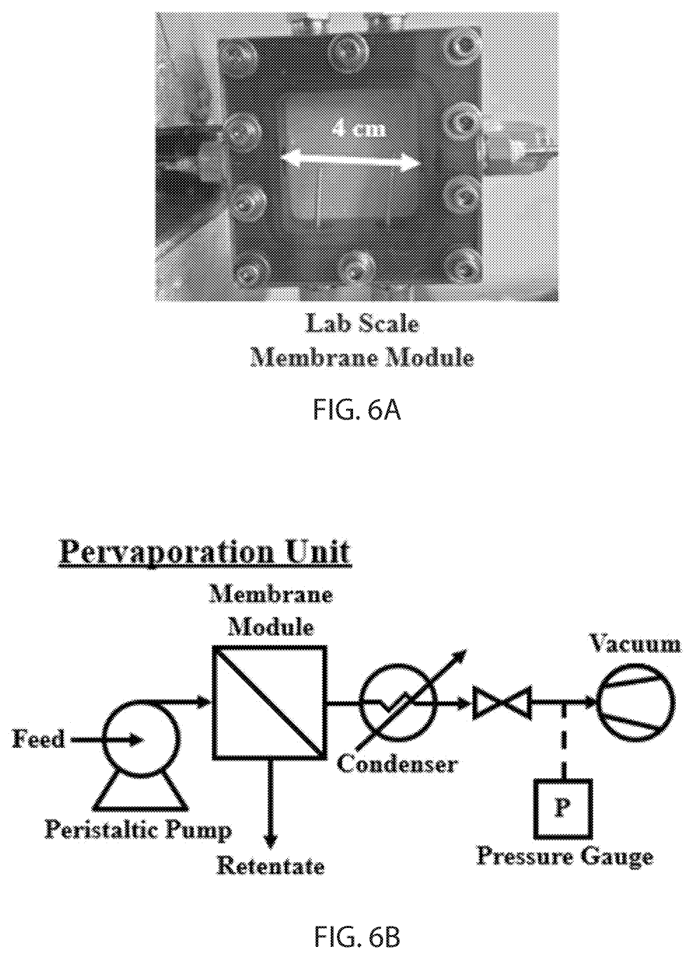

A schematic diagram of a lab-scale pervaporation system is presented in FIG. 6B. Each membrane is tested using deionized water for 30 minutes to verify that no leaks are present at an initial testing condition of a feed flow rate of 1 mL/s with a vacuum level of -0.9 bar. Very high water fluxes indicate the presence of membrane defects. Histidine buffer solution feeds ranging from 2 to 20 mM are tested to determine the effect of buffer concentration on pervaporation retentate.

After confirming that there are no leaks in the system, the feed is replaced with solutions of polyethylene glycol (MiraLAX®, molecular weight 3,350 g/mol) in deionized water. An initial concentration of 300 mg/mL is used to determine if preliminary membrane performance appears promising (e.g., flux > 1.4 L m⁻² h⁻¹). Then, test feeds are expanded with a range of concentrations between 25 and 1,000 mg polyethylene glycol / mL of water to determine flux and permeance, and to monitor for any flux decline as the feed becomes more concentrated. Note that polyethylene glycol has a solubility of ~61% in water (~1597 mg/mL); therefore, this is a reasonable range over which to test. The tests are conducted using feed solutions maintained at room temperature (20-24 ºC) and run the system for up to 24 hours. A goal is to achieve membranes with fluxes of ≥ 9-10 L m⁻² h⁻¹ (corresponding to permeances of ≥ 400 L m⁻² h⁻¹ bar⁻¹) for concentrated polyethylene glycol solutions. The equations of flux and permeance calculations are shown in FIG. 5.

Once it is shown that the membrane works well in extreme conditions (e.g., PEG feed solution has a concentration > 600 mg/mL), the membrane is tested with mAb feed. The feed solution is concentrated by a factor of 10. Tests are run for 10 hours to assess potential flux decline. A potential source of flux decline could be from the increased viscosity of the mAb solution as compared to the same concentration of polyethylene glycol. However, this issue is mitigated by adjusting the feed cross-flow rate in the system to alter the Reynolds number. Successful performance is achieving an average pervaporation flux in the mAb concentrating tests ≥ 50% of the pervaporation flux in polyethylene glycol tests, under the same operating conditions.

After achieving the membrane with ideal performance, the membrane is scaled up (e.g., 0.3 × 5 m) using Wellspring Co., Ltd.'s roll-to-roll casting system. This type of membrane is used to synthesize membrane modules that is compatible with the lab-scale system. The membrane modules are tested and evaluated to verify that they can achieve a flux of 200 kg/m²/hour.

### Example 4: Data Analysis

Performance is analyzed by measuring the concentration of analytes in the feed/retentate before and after an experiment, as well as in the permeate after an experiment. Flux and concentration factor are calculated by performing a standard mass balance on the system, using the measured captured permeate. Core performance is assessed based on flux, permeance, and rejection/retention. The calculation equations are shown in FIG. 5.

Characterization of the membrane surface is performed with scanning electron microscopy on samples of the membrane at various points. The consistency of the selective layer thickness and the absence of pores on the surface is verified. The selective layer is characterized by using Fourier transform infrared spectroscopy at various points on the membrane to confirm the successful synthesis of the thin-film composite membrane. Membranes are also examined after pervaporation experiments to determine if there is any irreversible deposition of PEG (or mAb) onto the membrane surface.

Some further aspects are defined in the following clauses:

Clause 1: A method for concentrating a biopharmaceutical feed stream comprising a biomolecule, the method comprising: (a) contacting a first side of a dense, non-porous membrane with the biopharmaceutical feed stream, wherein the feed stream comprises at least one biomolecule and a solvent; (b) maintaining a chemical potential gradient across the membrane such that at least a portion of the solvent is transported through the membrane via a solution-diffusion mechanism and exits a second side of the membrane as a vapor permeate; and (c) recovering a retentate stream from the first side of the membrane, wherein the retentate stream comprises the biomolecule at a concentration higher than that of the feed stream.

Clause 2: The method of Clause 1, wherein the chemical potential gradient is maintained by applying a vacuum to the second side of the membrane to create a vapor pressure differential.

Clause 3: The method of Clause 1 or Clause 2, wherein the biomolecule comprises a monoclonal antibody (mAb), a recombinant protein, or a nucleic acid.

Clause 4: The method of any one of the preceding Clauses 1-3, wherein the concentration of the biomolecule in the retentate stream is greater than 200 mg/mL, preferably greater than 300 mg/mL, and more preferably up to 600 mg/mL.

Clause 5: The method of any one of the preceding Clauses 1-4, wherein the dense, non-porous membrane comprises a sulfonated pentablock copolymer.

Clause 6: The method of any one of the preceding Clauses 1-5, wherein the sulfonated pentablock copolymer comprises polyisoprene, poly(t-butyl styrene), polystyrene, and poly(sulfonated styrene).

Clause 7: The method of any one of the preceding Clauses 1-6, wherein the membrane is a thin-film composite (TFC) membrane comprising an active selective layer having a thickness between 100 nm and 20 µm disposed on a porous support material.

Clause 8: The method of any one of the preceding Clauses 1-7, wherein the biopharmaceutical feed stream further comprises at least one non-volatile excipient or buffer, and wherein the method further comprises retaining the non-volatile excipient or buffer in the retentate stream alongside the concentrated biomolecule.

Clause 9: The method of any one of the preceding Clauses 1-8, further comprising a pre-concentration step using ultrafiltration or diafiltration prior to the pervaporation contacting step.

Clause 10: The method of any one of the preceding Clauses 1-9, wherein the method is performed as a continuous flow-through process or a feed-and-bleed process.

Clause 11: A system for the concentration of biopharmaceutical products, the system comprising: a feed reservoir containing a feed stream comprising a biomolecule at an initial concentration; a pervaporation module comprising a dense, non-porous membrane separating a feed side from a permeate side; a vacuum source in fluid communication with the permeate side of the membrane configured to maintain a vapor pressure differential across the membrane; and a condenser configured to capture solvent vapor exiting the permeate side; wherein the system is configured to concentrate the biomolecule to a final concentration of at least 200 mg/mL at a volumetric flux of at least 0.1 L m⁻² h⁻¹.

Clause 12: The system of Clause 11, wherein the dense, non-porous membrane exhibits a water permeance of at least 300 L m⁻² h⁻¹ bar⁻¹.

Clause 13: The system of Clause 11 or Clause 12, further comprising an ultrafiltration unit positioned upstream of and in fluid communication with the pervaporation module.

Clause 14: A thin-film composite pervaporation membrane specifically configured for biopharmaceutical concentration, comprising: (a) a porous support layer comprising polysulfone or polyethersulfone; and (b) a dense, non-porous active layer disposed on the support layer, the active layer comprising a sulfonated pentablock copolymer; wherein the membrane is characterized by the ability to retain >99% of a monoclonal antibody in a feed stream while maintaining a solvent flux of at least 1.0 L m⁻² h⁻¹ under a vacuum of -0.9 bar.

Clause 15: A concentrated biopharmaceutical formulation produced by the method of claim 1, comprising a monoclonal antibody at a concentration of 200 mg/mL to 600 mg/mL and at least one concentrated excipient, wherein the formulation is suitable for subcutaneous injection.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method for concentrating a biopharmaceutical feed stream comprising a biomolecule, the method comprising:
(a) contacting a first side of a dense, non-porous membrane with the biopharmaceutical feed stream, wherein the feed stream comprises at least one biomolecule and a solvent;
(b) maintaining a chemical potential gradient across the membrane such that at least a portion of the solvent is transported through the membrane via a solution-diffusion mechanism and exits a second side of the membrane as a vapor permeate; and
(c) recovering a retentate stream from the first side of the membrane, wherein the retentate stream comprises the biomolecule at a concentration higher than that of the feed stream.

2. The method of claim 1, wherein the chemical potential gradient is maintained by applying a vacuum to the second side of the membrane to create a vapor pressure differential.

3. The method of claim 1, wherein the biomolecule comprises a monoclonal antibody (mAb), a recombinant protein, or a nucleic acid.

4. The method of claim 3, wherein the concentration of the biomolecule in the retentate stream is greater than 200 mg/mL, preferably greater than 300 mg/mL, and more preferably up to 600 mg/mL.

5. The method of claim 1, wherein the dense, non-porous membrane comprises a sulfonated pentablock copolymer.

6. The method of claim 5, wherein the sulfonated pentablock copolymer comprises polyisoprene, poly(t-butyl styrene), polystyrene, and poly(sulfonated styrene).

7. The method of claim 1, wherein the membrane is a thin-film composite (TFC) membrane comprising an active selective layer having a thickness between 100 nm and 20 µm disposed on a porous support material.

8. The method of claim 1, wherein the biopharmaceutical feed stream further comprises at least one non-volatile excipient or buffer, and wherein the method further comprises retaining the non-volatile excipient or buffer in the retentate stream alongside the concentrated biomolecule.

9. The method of claim 1, further comprising a pre-concentration step using ultrafiltration or diafiltration prior to the pervaporation contacting step.

10. The method of claim 1, wherein the method is performed as a continuous flow-through process or a feed-and-bleed process.

11. A system for the concentration of biopharmaceutical products, the system comprising:
a feed reservoir containing a feed stream comprising a biomolecule at an initial concentration;
a pervaporation module comprising a dense, non-porous membrane separating a feed side from a permeate side;
a vacuum source in fluid communication with the permeate side of the membrane configured to maintain a vapor pressure differential across the membrane; and
a condenser configured to capture solvent vapor exiting the permeate side;
wherein the system is configured to concentrate the biomolecule to a final concentration of at least 200 mg/mL at a volumetric flux of at least 0.1 L m⁻² h⁻¹.

12. The system of claim 11, wherein the dense, non-porous membrane exhibits a water permeance of at least 300 L m⁻² h⁻¹ bar⁻¹.

13. The system of claim 11, further comprising an ultrafiltration unit positioned upstream of and in fluid communication with the pervaporation module.

14. A thin-film composite pervaporation membrane specifically configured for biopharmaceutical concentration, comprising:
(a) a porous support layer comprising polysulfone or polyethersulfone; and
(b) a dense, non-porous active layer disposed on the support layer, the active layer comprising a sulfonated pentablock copolymer;
wherein the membrane is characterized by the ability to retain >99% of a monoclonal antibody in a feed stream while maintaining a solvent flux of at least 1.0 L m⁻² h⁻¹ under a vacuum of -0.9 bar.

15. A concentrated biopharmaceutical formulation produced by the method of claim 1, comprising a monoclonal antibody at a concentration of 200 mg/mL to 600 mg/mL and at least one concentrated excipient, wherein the formulation is suitable for subcutaneous injection.
